(19) United States
(12) Patent Application Publication
(10) Pub. No.: US 2013/0337580 A1
(43) Pub. Date: Dec. 19, 2013

(54) Immunoassay Device and Method

(71) Applicant: Kiamars Hajizadeh, Falmouth, ME (US)

(72) Inventor: Kiamars Hajizadeh, Falmouth, ME (US)

(21) Appl. No.: 13/886,679

(22) Filed: May 3, 2013

Related U.S. Application Data
(60) Provisional application No. 61/641,966, filed on May 3, 2012.

(51) Int. Cl. G01N 33/53

(52) U.S. Cl. CPC G01N 33/53
USPC 436/501; 422/69

(57) ABSTRACT

Devices, methods and kits for the detection of analytes in liquid samples. A device for conducting an assay to determine the presence or amount of an analyte in a fluid sample having a flow path matrix that facilitates fluidic flow and a fluid impermeable barrier. The flow path includes a first region on a top side of the barrier that facilitates fluid flow in a first direction and comprises a sample entry area and a second region on a bottom side of the barrier that facilitates fluid flow in a second direction substantially opposite the first direction and comprises a detection zone. Methods using the devices. Kits including the devices and various reagents necessary for conducting the methods.

### BACKGROUND

1. Field of the Invention

The invention relates to test devices and methods for performing assays to determine the presence or amount of an analyte in a sample.

2. Description of Related Art

Lateral flow type devices for the detection and quantification of an analyte of interest in a fluid sample are well known. For example, such devices are described in U.S. Pat. Nos. 3,799,742; 3,811,840; 3,645,687; 4,435,504; 4,094,647; 3,246,339; 4,366,241; 3,888,629, and 5,750,333, each of which is incorporated by reference herein in its entirety. Generally, the devices include a solid phase fluid permeable flow path through which fluid travels by capillary action having immobilized thereon various capture reagents for the analyte (or analogue thereof) or conjugates involving binding partners for the analyte and members of signal producing systems (e.g., a label). The various assay formats used with these devices are well known for the direct or indirect detection of the analyte of interest in the test sample.

Sample volume is one way of controlling the sensitivity of the assay, since enough sample is necessary in order to provide a detectable quantity of the analyte. For a small volume sample, one way to control sensitivity of the assay is to control how the sample is added to the device. Numerous ways are known for adding sample to solid phase flow paths of lateral flow devices. For instance, sample may be added directly to the flow path or to a sample holder. Such devices are described in U.S. Pat. Nos. 4,943,522, 4,956,302; 5,939,331; 6,468,474; 6,607,922; 6,686,208; and 6,706,539, each of which is incorporated by reference herein in its entirety. In addition to sensitivity, the ease of operator use and the minimization of steps are commercially desirable features.

Accordingly, the inventors have identified a need in the art for an assay device that decreases sample and reagent handling while maintaining assay sensitivity

### SUMMARY

In one aspect, the invention is directed to a device for conducting an assay to determine the presence or amount of an analyte in a fluid sample. The device includes a flow path matrix that facilitates fluidic flow and a fluid impermeable barrier. The flow path matrix includes a first region on a top side of the barrier that facilitates fluid flow in a first direction and comprises a sample entry area, and a second region on a bottom side of the barrier that facilitates fluid flow in a second direction substantially opposite the first direction and comprises a detection zone. In various embodiments, the device also includes a third region in fluid communication with the first and second region. Also, the flow path may include a mobilizable conjugate reagent that binds to the analyte, wherein the conjugate reagent comprises a label or a first binding partner for a second binding partner comprising a label. In addition, the detection zone can include an immobilized binding partner for the analyte or an immobilized analog of the analyte. In further embodiments, the flow path has total length of less than 2 cm.

In another aspect, the invention is directed to a device for conducting an assay to determine the presence or amount of an analyte in a fluid sample. The device includes a flow path matrix that facilitates fluidic flow. The matrix partially circumscribes a planar fluid impermeable barrier having a top side and a bottom side, and includes a sample entry area on the top side of the fluid impermeable barrier and a detection zone on the bottom side of the fluid impermeable barrier. In various embodiments, the flow path comprises a mobilizable conjugate reagent that binds to the analyte, wherein the conjugate reagent comprises a label or a first binding partner for a second binding partner comprising a label. The detection zone can include an immobilized binding partner for the analyte or an immobilized analog of the analyte. And the flow path may have a total length of less than 2 cm.

In a further aspect, the invention is directed to a method for conducting an assay to determine the presence or amount of an analyte in a fluid sample. The method includes contacting the fluid sample with the device of the invention and detecting the presence or amount of a signal associated with the label in the detection zone.

Still further, the invention is directed to a method for conducting an assay to determine the presence or amount of an analyte in a fluid sample. The method includes contacting the fluid sample with the device of the invention and detecting the presence or amount of a signal associated with the label in the detection zone.

Still further, the invention is directed to a test kit for conducting an assay to determine the presence or amount of an analyte in a fluid sample, the kit comprising a device of the invention and a labeled specific binding reagent.

In yet another embodiment, the invention is directed to an apparatus for use in the detection of an analyte in a sample. The apparatus includes a housing that retains the device of the invention, a sample application window corresponding to the sample application area, and a detection window corresponding to the detection area. The apparatus may be configured to be inserted into an analyzer comprising a reader for reading a signal from detection zone through the detection window.

### BRIEF DESCRIPTION OF THE FIGURES

FIGS. 1A-1D. are cross sectional views of various embodiment of a device of the invention.

FIG. 2. shows top and bottom views of various embodiments of the device of the invention.

FIGS. 3A and 3D. show the results of analyses conducted with various embodiments of the device of the invention.

FIG. 4. is a graph showing results of an analyses conducted with an embodiment of the device of the invention.

FIG. 5 shows embodiments of the device of the invention used in the Catalyst® Chemistry Analyzer to test the dose sensitive response of anti-FPL conjugated colloidal gold.

### DESCRIPTION

The invention relates to test devices and methods for performing assays to determine the presence or amount of an analyte in a sample. The invention is useful for a wide variety of assays, both ligand-based and non-ligand-based. Applicable ligand-based methodologies may include, but are not limited to, competitive immunoassays, non-competitive or so-called sandwich technique immunoassays, and blocking assays. The use of the invention is not limited to any particular analyte. The embodiments described herein are solely for illustrative purposes but are not intended to limit the scope of the invention to any particular set of binding partners or assay format.

In one aspect, the invention is directed to a one-step assay based on flow dynamics through membranes assembled to form a unitized test device in a very small space. In one embodiment, the sample is applied at the top of the device and flows horizontally in a first direction through a flow path matrix. Flow continues vertically through a region connecting the top portion of the matrix to a bottom section where the sample flows in a second direction essentially opposite of the first direction. The bottom section includes an immobilized member of the binding pair that can bind an analyte or other sample components that allow for the detection the presence or amount of the analyte. The result of the test can be read by an analytical instrument or visually from the bottom of the device at the detection zone.

In one aspect, the invention is directed to a devices, methods and kits for determining the presence or amount of an analyte in a liquid sample. In one aspect, the invention is directed to a device for conducting an assay to determine the presence or amount of an analyte in a fluid sample. The device includes a single flow path matrix that facilitates fluidic flow by capillary action. The flow path has three directions with a first region for sample entry and having a first direction, a second region having a second direction substantially opposite the first direction, and a third region in fluid communication with the first and second regions to allow for fluid flow between the first and second regions. The second region includes a detection zone having an immobilized capture reagent. Additionally, there is a fluid impermeable barrier between the first region and the second region in order to prevent cross-talk between the first region and the second region and block any background color from the first region penetrating the second region.

Before describing the present invention in detail, a number of terms will be defined. As used herein, the singular forms “a,” “an”, and “the” include plural referents unless the context clearly dictates otherwise.

By “analyte” is meant a molecule or substance to be detected. For example, an analyte, as used herein, may be a ligand, which is mono- or polyepitopic, antigenic or haptenic; it may be a single compound or plurality of compounds that share at least one common epitopic site; it may also be a receptor or an antibody.

An “analyte analog” is a specific derivative of the target analyte that may be optionally attached, either covalently or non-covalently, to other chemical species (e.g., a label). The analyte analog may be used, for example, to compete with the analogous target analyte for binding to the specific binding partner (i.e., competition assay). Where the modification of the analyte provides means to join the analyte analog to another molecule, or where the analyte has a functionality that is used to bind directly to another molecule, the analyte portion of the conjugate will be referred to as an analyte analog.

A “specific binding partner” or “binding partner” is a molecule, such as an oligonucleotide, or a receptor, binding protein, antibody or antibody fragment, or enzyme (which binds to its substrate), that possesses the ability to interact with another molecule in a highly specific polar and spatial manner.

An “antibody” is an immunoglobulin, or a derivative or fragment thereof, that is capable of specifically binding to an antigen in a receptor-ligand based reaction. The antibody or fragment may be polyclonal or monoclonal, or native or chimeric.

A “label” is any molecule that is bound (via covalent or non-covalent means, alone or encapsulated) to another molecule or solid support and that is chosen for specific characteristics that allow detection of the labeled molecule. Generally, labels are comprised of, but are not limited to, the following types: particulate metal and metal-derivatives, radioisotopes, catalytic or enzyme-based reactants, chromogenic substrates and chromophores, fluorescent and chemiluminescent molecules, and phosphors. The utilization of a label produces a signal that may be detected by means such as detection of electromagnetic radiation or direct visualization, and that can optionally be measured.

By “immobilized capture reagent” is meant a molecule that is bound covalently or non-covalently to a solid phase matrix or support and that has a specific affinity for an analyte of interest, analyte analogue, or other binding partners for the immobilized capture reagent. Preferably, the affinity arises by virtue of the immobilized reagent possessing a complementary three-dimensional structure to the analyte, analogue, or other binding partner. For example, as seen in the relationship between an enzyme and a substrate or an antigen and an antibody. Within a given pair including the analyte, either member may be considered to be the analyte or the capture reagent. The definition serves only to differentiate the members of the binding pair, i.e. the partner that is immobilized from the partner that is not immobilized

A “labeled specific binding reagent” is a non-immobilized substance that specifically binds to the analyte (or analogue) and is bound, directly or indirectly, to a label.

A “sample” refers to an aliquot of any matter containing, or suspected of containing, an analyte of interest. For example, samples include biological samples, such as samples taken from animals (e.g., saliva, whole blood, serum, and plasma, urine, tears and the like), cell cultures, plants, etc.; environmental samples (e.g., water); and industrial samples. While the device of the invention is appropriate for use with undiluted liquid samples, samples may be prepared prior to use in the methods of the invention. For example, samples may require diluting, filtering, centrifuging or stabilizing prior to use with the invention. For the purposes herein, “sample” refers to the either the raw sample or a sample that has been prepared.

“Fluid flow matrix” refers to a solid phase matrix capable of providing capillary mediated lateral flow to a liquid test sample and/or liquid reagents. Generally, the porous carrier matrix can be selected from any available material having appropriate thickness, pore size, lateral flow rate, and color. Lateral flow refers to liquid flow in which all of the sample and reagents components are carried at substantially equal rates and with relatively unimpaired flow laterally through the matrix, as opposed to the preferential retain of one or more components of the liquid, such as a chromatographic separation of the sample components.

The fluid flow matrix material preferably possesses the following characteristics: (1) low non-specific affinity for sample materials and labeled specific binding reagents, (2) ability to transport a liquid by capillary action over a distance with a consistent liquid flow across the matrix, and (3) readily binding to immobilized specific binding reagents, (e.g., by covalent or non-covalent attachment or by physical entrapment). Materials possessing these characteristics include woven fabrics or fibrous mats composed of synthetic or natural fibers (e.g., glass or cellulose-based materials or thermoplastic polymers, such as, polyethylene, polypropylene, or polyester); sintered structures composed of particulate materials (e.g., glass or various thermoplastic polymers); or cast membrane films composed of nitrocellulose, nylon, polysulfone or the like (generally synthetic in nature). The invention may utilize a flow matrix composed of sintered, fine particles of polyethylene, commonly known as porous polyethylene; preferably, such materials possess a density of between 0.35 and 0.55 grams per cubic centimeter, a pore size of between 5 and 40 microns, and a void volume of between 40 and 60 percent. Particulate polyethylene composed of cross-linked or ultra-high molecular weight polyethylene is preferable. A flow matrix composed of porous polyethylene possesses all of the desirable features listed above, and in addition, is easily fabricated into various sizes and shapes.

The fluid flow matrix may be made from a material which has a low affinity for the analyte and test reagents. This is to minimize or avoid pretreatment of the test matrix to prevent nonspecific binding of analyte and/or reagents. However, materials that require pretreatment may provide advantages over materials that do not require pretreatment. Therefore, materials need not be avoided simply because they require pretreatment. Hydrophilic matrices generally decrease the amount of non-specific binding to the matrix.

An example of a suitable fluid flow matrix in which lateral flow occurs is the high density or ultra-high molecular weight polyethylene sheet material manufactured by Porex Technologies Corp. of Fairburn, Ga., USA. This material is made from fusing spherical particles of ultra-high molecular weight polyethylene (UHWM-PE) by sintering. This creates a porous structure with an average pore size of eight to 20 microns, depending on the size of the particles (20 to 60 microns, respectively). The polyethylene surface is treated with oxygen plasma and then coated with alternating layers of polyethylenimine (PEI) and poly acylic acid (PAA) to create surfactant-free hydrophilic surface having wicking rate of 0.01-0.5 cm/s.

While matrices made of polyethylene have been found to be highly satisfactory, lateral flow materials formed of other olefin or other thermoplastic materials, e.g., polyvinyl chloride, polyvinyl acetate, copolymers of vinyl acetate and vinyl chloride, polyamide, polycarbonate, polystyrene, etc., can be used. Examples of suitable materials include FUSION 5™ from Whatman Inc. (Piscataway, N.J.); Magna Nylon Supported Membrane from GE Osmonics (Minnetonka, Minn.); Novylon Nylon Membrane from CUNO Inc (Meriden, Conn.); and Durapore Membrane from Millipore (Billerica, Mass.).

The matrix materials may be slit, cut, die-cut or punched into a variety of shapes prior to incorporation into a device. Examples of alternative shapes of the matrix include circular, square/rectangular-shaped, flattened ellipse shaped or triangularly shaped. While not a focus of the invention, if desired, biological reagents may be applied to the materials before or after forming the desired shape. Biological reagents may be attached to the materials by any available method, for example, either by passively, diffusively, non-diffusively, by absorption, or covalently, depending upon the application and the assay.

In one aspect, the fluid flow matrix has an open pore structure with an average pore diameter of 1 to 250 micrometers and, in further aspects, about 3 to 100 micrometers, or about 10 to about 50 micrometers. The matrixes are from a few millimeters (0.001 inches) to several millimeters in thickness, typically in the range of from 5 or 10 millimeters and up to 200 millimeters; in one embodiment the matrix thickness is 0.5 millimeters to 2.0 millimeters. In one aspect, the length of the matrix is from 1.0 centimeter to 2.0 centimeters, and the width of the matrix is 0.3 centimeters to 1.0 centimeter. The matrix should be translucent to allow for the visualization or photometric determination of the light and or color throughout the thickness of the matrix. The matrix may be backed with a generally water impermeable layer, or may be totally free standing.

A “conjugate reagent” is specific binding partner or binding partner of the analyte and is a molecule, such as a receptor, binding protein, antibody or antibody fragment, or enzyme (which binds to its substrate), that possesses the ability to interact with another molecule in a highly specific polar and spatial manner. The conjugate reagent also includes a label, or a moiety that is capable of binding to a label. For example, the conjugate reagent may include a specific binding partner for the analyte attached to a label. The attachment of the binding partner to the label may be accomplished covalently or non-covalently by any procedure well known to those of skill in the art. The label may be indirectly attached to the binding partner, such as through a biotin/avidin interaction, where the label is attached to biotin and the binding partner is attached to streptavidin (or vice versa).

By “immobilized binding reagent” is meant a molecule which is bound to a solid support and which has a specific affinity for an analyte of interest. Preferably, the affinity arises by virtue of the reagent possessing a complementary three-dimensional structure to the analyte, for example, as seen in a specific binding relationship such as the relationship between an enzyme and a substrate or an antigen and an antibody. Within a given pair, either member may be considered to be the analyte or the binding reagent. The definition serves only to differentiate the component to be detected in the sample (the analyte) from the reagent included in the device or method (an analyte binding partner). The Biojet XYZ-3050 in one example of a device that could be used to apply the binding reagent to a membrane.

Turning now to the invention in more detail, in one embodiment, the invention is directed to a device for conducting an assay to determine the presence or amount of an analyte in a fluid sample. A flow path matrix facilitates fluidic flow within the device, which also includes a fluid impermeable barrier. The flow path has a first region on a top side of the barrier that facilitates fluid flow in a first direction and includes a sample entry area. A second region on a bottom side of the barrier facilitates fluid flow in a second direction substantially opposite the first direction and includes a detection zone. A third region is in fluid communication with the first and second regions to allow for fluid flow between the first and the second regions.

The fluid impermeable barrier is preferably opaque and can be any inert material that is compatible with the reagents used in the assay. Flow path matrix materials can be mounted on the barrier in any known manner, including adhesives such as double-sided tape. In addition, the component of the device can be mounted in a housing without the use of adhesives between the components.

In another embodiment, the flow path is a layer of matrix material that partially circumscribes a planar fluid impermeable barrier having a top side and a bottom side. The matrix has a sample entry area on the top side of the fluid impermeable barrier and a detection zone on the bottom side of the fluid impermeable barrier.

In some embodiments, the flow path has total length of less than 2 cm. This facilitates the use of the device in analytical devices that can detect a signal produced in the detection zone. See, for example, US Patent Publication 2010/0254854, which is incorporated herein by reference in its entirety.

In one aspect of the invention, a detector reagent is provided to detect a labeled specific binding reagent captured in the detection zone. The detector reagent participates with the label to produce one or more measurable signals to indicate the presence or quantity of the analyte in the sample.

In another aspect of the invention, the device can be used in a homogeneous assay format where no reagent is immobilized to a solid phase (e.g., EMIT (enzyme multiplication immunoassay technique), CEDIA (Cloned enzyme donor immunoassay), ARIS (apo-enzyme reactivation immunoassay system), and EIHIA (enzyme inhibitory homogeneous immunoassay).

When the fluid sample is added to the first region, the conjugate reagent is solubilized and, depending upon the assay format, the conjugate reagent may specifically bind to an analyte in the sample to form a complex of the analyte and the conjugate reagent. In another aspect, the conjugate reagent includes an analyte analog, which does not complex with the analyte.

In one aspect, the first region contains a solubilizable conjugate reagent, and the flow path matrix includes an immobilized analyte binding partner in a detection zone in the second region of the matrix spaced from the first region. The conjugate reagent is solubilized by adding the sample to the first region. The sample and conjugate reagent are allowed to migrate to the second region, and unbound sample and conjugate reagent are washed from the second region. The presence of the conjugate reagent in the detection zone is detected.

In various aspects of the invention, the dried conjugate reagent is present in a sample application element that facilitates addition of the sample to the first region. See, e.g., U.S. Pat. No. 7,816,822, which is incorporated by reference herein in its entirety. The conjugate reagent is readily solubilized by a sample solution, which is present in the element for only a short amount of time, i.e., the time it takes for the sample to drain from the element into the matrix. In one aspect, the conjugate reagent is immediately solubilized upon the addition of the sample solution to the element.

In another aspect, the third region, positioned in between the first and second regions of the flow path, contains a soluble conjugate reagent. The conjugate reagent is solubilized by flow of the sample through the third region. The sample and conjugate reagent are allowed to migrate to the second region, and unbound sample and conjugate reagent are washed from the second region. The presence of the conjugate reagent in the second region is detected.

In various embodiments of the invention, the third region of the device includes portions of the first and second regions, and is not a separate piece of matrix material. In these embodiments, the fluid impermeable barrier is not completely coextensive with the first and second regions, which creates a region that allows for fluid transfer directly from the first regions to the second region. The first and second regions, therefore, create a third region wherein fluid is transferred through this region from the first region to the second region. In some embodiments of the invention the third region is created by physical contact between the first region and the second region alleviating the need for a third region. The third region may be made of a porous membrane, glue or a wet-able polymer connecting the first region and the second region, a combination of the two, or may be nothing.

In further aspect, the sample and the conjugate are mixed before being added to the first region. The flow path matrix includes an immobilized analyte binding partner in the second region of the matrix that is horizontally and vertically spaced from the first region. The conjugate reagent is solubilized by addition of the sample. The sample and conjugate reagent are applied to the first region and allowed to migrate to the second region, and unbound sample and conjugate reagent are washed from the second region. The presence of the conjugate reagent in the second region is detected.

In one embodiment, in order to facilitate the capillary flow of the entire sample and the fluid reagent through the detection zone, the device includes an absorbent reservoir to absorb excess liquid. The excess fluid capacity of the reservoir ensures the flow of the entire liquid sample through the detection zone, and the ability of the liquid reagent to wash the detection zone of unbound analyte and conjugate reagent.

Materials suitable for use as an absorbent reservoir are preferably highly absorbent, provide capacity in excess of the volume of the fluid sample plus the added liquid reagents, and are capable of absorbing liquids from the flow matrix by physical contact as the sole means of fluid transfer between the two materials. A variety of materials and structures are consistent with these requirements. Fibrous structures of natural and synthetic fibers such as cellulose and derivatized cellulose (e.g., cellulose acetate) are preferred for this use. The fibers of the material may be oriented along a particular axis (i.e., aligned), or they may be random. An embodiment of the invention utilizes non-aligned cellulose acetate fibers of density range 0.1 to 0.3 grams per cubic centimeter and void volume of 60 to 95 percent. Particularly preferred materials include American Filtrona Corporation R-13948 Transorb Reservoir (Richmond, Va.) and Drikette® desiccant paper (Multisorb Technologies; Buffalo, N.Y.).

Any or all of the above embodiments of the invention may be provided as a kit. In one example, a kit would include a device of the invention complete with specific binding reagents, for example, the dried conjugate reagent and immobilized binding reagents, as well as wash reagent and detector reagent. Positive and negative control reagents may also be included, if desired or appropriate. In addition, other additives may be included, such as stabilizers, buffers, and the like. The relative amounts of the various reagents may be varied widely, to provide for concentrations in solution of the reagents that substantially optimize the sensitivity of the assay.

The conjugate reagent may also be labeled with an enzyme. In such a case, the detector reagent includes a substrate that produces a detectable signal upon reaction with the enzyme-labeled conjugate-analyte complex at the detection zone. In another aspect, the conjugate reagent has a visible label, such as a colloidal gold. The liquid wash reagent removes any unbound visible label from the detection zone. The conjugate reagent may be labeled with a radioactive, fluorescent, or light-absorbing molecule. In such a case, the detector reagent acts merely as a wash solution facilitating detection of complex formation at the detection zone by washing away unbound labeled reagent.

In another embodiment, an analyte analog may be used, for example, to compete with the analogous target analyte for binding to the specific binding partner (that is, competition assay).

In another aspect the invention is directed to methods for conducting an assay to determine the presence or amount of an analyte in a fluid sample. The methods include, for example, competitive immunoassays and non-competitive or so-called sandwich technique immunoassays. In a sandwich assay, the detection zone of the device includes an analyte specific binding partner immobilized thereon. The conjugate reagent includes a second binding partner for the analyte that includes a label. As described above, the conjugate reagent can be present on the device in dried form, or it can be added to the sample prior to the addition of the sample to the device. After the sample is added to the sample application zone, the amount of signal associated with the label of the conjugate reagent is detected in the detection zone.

For competitive immunoassays, the detection zone includes either an analyte analog a binding partner for the analyte. When the detection zone includes an analyte analog, the conjugate reagent includes a binding partner for the analyte. Accordingly, analyte in the sample competes with the analyte analog for binding to the conjugate reagent. As the amount of analyte increases, the signal associated with the label at the detection zone decreases because the conjugate reagent is prevented from binding to the analyte analog in the detection zone.

Alternatively, for competitive assays, when the detection zone includes a specific binding partner the analyte, the conjugate reagent includes an analyte analog that competes with the analyte for binding to the detection zone. As the amount of analyte increases, the signal associated with the label at the detection zone decreases because the conjugate reagent is prevented from binding to the analyte analog in the detection zone.

Binding partners can be bound to the detection zone and the label directly, for example through covalent interaction, or indirectly, for example though a separate set of binding partners (biotin/avidin, protein A/G). These techniques are well known to one of ordinary skill in the art.

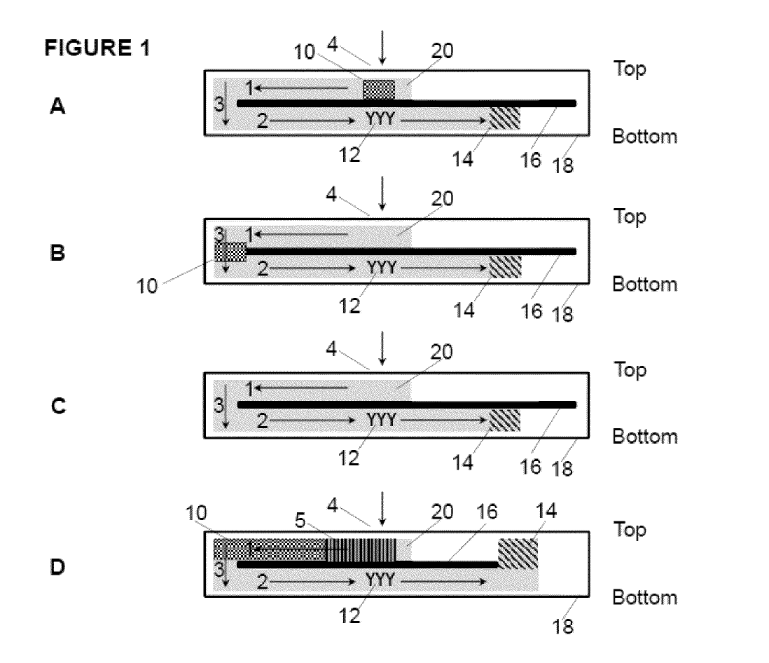

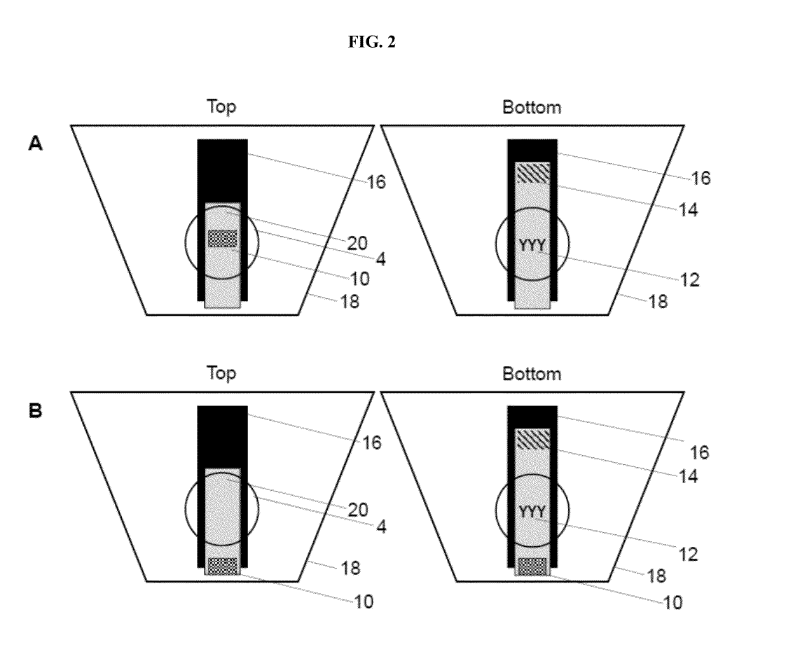

FIGS. 1 and 2 provide examples of embodiments (A, B, C, and D) of the device of the invention. A fluid flow path matrix 20 for use in the device is shown comprising: a first region 1 at a top portion of the matrix having a first direction comprising a sample entry area 4; a second region 2 at a bottom portion of the matrix having a direction essentially opposite of the first direction and comprising a detection zone 12 with immobilized capture reagents (depicted as Y); and a third region 3 in fluid communication with the first and second regions. A conjugate reagent 10 (e.g., a labeled specific binding reagent that has been deposited and dried) may be in the first or third region as depicted in embodiments A and B respectively, may be in the second region, or may be added directly to the sample before entering the device as depicted in embodiment C of FIG. 1. In embodiment D of FIG. 1, the device includes a sample pad 5 in fluid communication with the first region 1 that includes conjugate reagent 10.

In one example, to carry out a binding assay using such a device, fluid sample is applied through a sample entry area 4 to the first region 1 of the flow matrix 20. The sample flows through the conjugate reagent 10. Contact of the conjugate reagent with the fluid sample results in dissolution of the labeled specific binding reagent into the sample, allowing sample analyte to bind to the labeled specific binding reagent. Positioning of the conjugate reagent 10 adjacent to the sample entry area 4 can increase the quantity of sample that contacts the dried reagent (FIG. 1A, D). In an alternative embodiment, the conjugate reagent can be in the third region (FIG. 1B), in the second region, or added to the sample before the sample is added to the device (FIG. 1C).

Sample and conjugate reagent are drawn, by capillary action, into the first direction of the first region 1 of the fluid flow matrix 20 and transported in the first direction towards the third region 3 which is in fluid communication between the first 1 and second 2 regions. The second region 2 carries the conjugate reagent and sample past the detection zone 12 where capture reagents have been immobilized. At the detection zone 12, all binding species are present (i.e., sample, labeled specific binding reagent, and immobilized capture reagent). Fluid flow continues through the second region until the fluid front reaches a desired position on the flow matrix, such as an absorbent reservoir 14. The absorbent reservoir is positioned toward one end of the matrix 20 so as to draw the fluid out of the matrix within the device. A wash reagent may be used to transport unbound sample and unbound labeled specific binding reagent along the fluid flow matrix 20 and away from the detection zone 12. In addition, the absorbent reservoir 14 is preferably of sufficient size to accommodate the total volume of sample as well as all added liquid reagents, that is, sample, detector reagent and/or wash reagent.

In one aspect, the detection zone 12 of the second region is seen from the outside of a clear housing 18, allowing ready detection of assay results. In all embodiments, the first region is separated from the second region by an impermeable barrier 16, which may be opaque to aid in detection of the labeled reagent.

In another aspect, the housing 18 is suitable for use with an automated or semi-automated analyzer, such as the Catalyst Dx® Chemistry Analyzer (IDEXX Laboratories). The housing can include a window that allows access to the sample application zone and a second window for allowing the analyzer to detect a signal in the detection zone. In particular embodiments, the flow path matrix has a length of less than 5 cm, for example less than 4 cm, less than 3 cm, less than 2 cm or less than 1 cm. A short matrix length allows, for example, the miniaturization of the housing. In addition, the sample application zone and detection zone can be sized to accommodate a small housing, for example a housing with a detection window of less than 5 mm, for example 3.5 mm. Accordingly, the detection zone could be readily visualized by the analytical instrument through the detection window.

In another embodiment, the first region, the second region, and the third region can be a single piece of matrix material, or it can be pieced together out of two or three pieces. The relative sizes of fluid flow matrix can vary and the proportions depicted in the figures herein are for illustrative purposes only. In an additional embodiment, the flow path may be a layer of matrix material partially circumscribing a planar fluid impermeable barrier having a top side and a bottom side, wherein the matrix includes the sample entry area on the top side of the fluid impermeable barrier and a detection zone on the bottom side of the fluid impermeable barrier.

When a homogenous assay format is used, separation of bound from free binding components is not required and therefore no binding partners are required in the detection zone. The binding member and analyte of the assay mixture will simply travel as a band through the flow matrix and through the detection zone where the peak of the band can be measured.

The following are provided for exemplification purposes only and are not intended to limit the scope of the invention described in broad terms above. All references cited in this disclosure are incorporated herein by reference.

### EXAMPLES

### Example 1

### Detection of THC on Catalyst Dx® Chemistry Analyzer at 560 nm

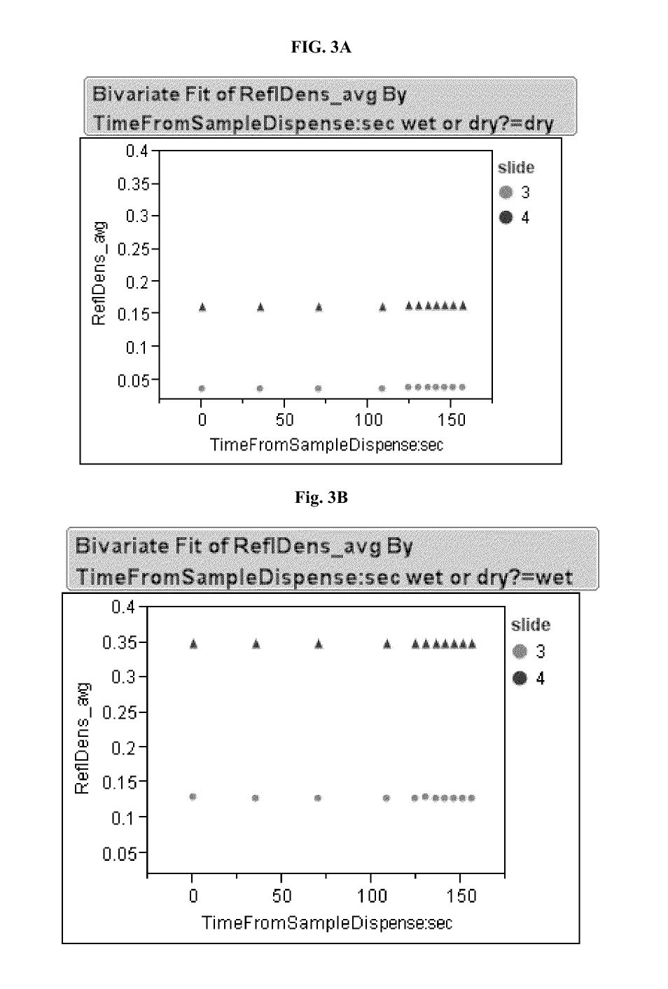

Feasibility was shown using a gold impregnated conjugate pad containing dried anti-THC (Tetrahydrocannabinol) gold conjugate. The conjugated pad was assembled into the device. A negative and a positive THC sample was applied to the upper side of a device and the assay monitored visually. A negative THC forms a strong red line (triangles) and a positive sample forms no line (circles) as expected for a competitive immunoassay. The slides developed in this manner were used in a Catalyst Dx® Chemistry Analyzer (IDEXX Laboratories; Westbrook, Me.) and read at 560 nanometers on the instrument while still wet (FIG. 3B) and after they were dried (FIG. 3A). The instrument detects the difference between gold intensities whether slides are dry (FIG. 3A) or wet (FIG. 3B) as shown in FIG. 3.

### Example 2

### Real Time Detection of Red Fluid at 560 nm

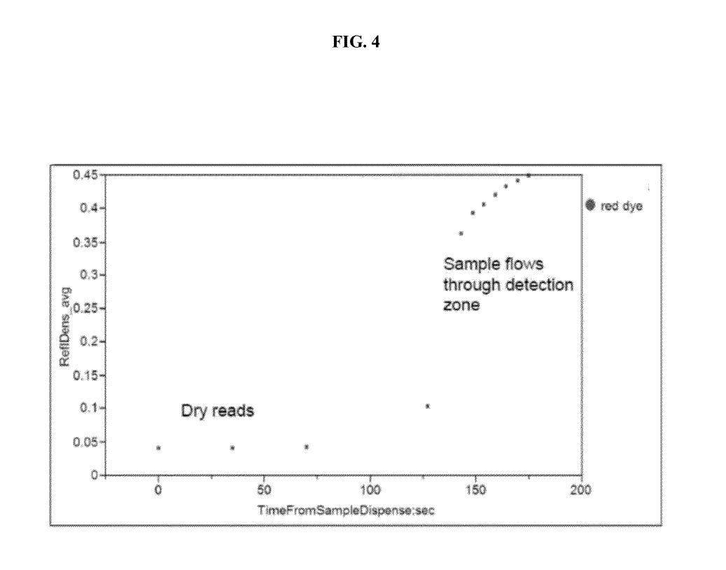

The assembled device was tested on an optical read instrument (Catalyst Dx® Chemistry Analyzer) with a red dye applied on top of the device. FIG. 4 shows the progress curve starting from dry reads before the red dye is applied to the application area of the device. After adding red dye as sample, the red dye migrates through the first region, around a double sided opaque membrane that prevents communication between the upper first region and the lower second region, to the detection zone where the detector is situated underneath the device. After the red dye goes through the vertical flow junction of the third region, it flows through the second region moving in the opposite direction of the upper first region until it reaches the detection zone. At this point the red dye is in the detection zone window and the color reflectance is detected by the optical read instrument at 560 nanometers.

### Example 3

### Dose Sensitive Response of Anti-FPL Conjugated Colloidal Gold to FPL Antigen

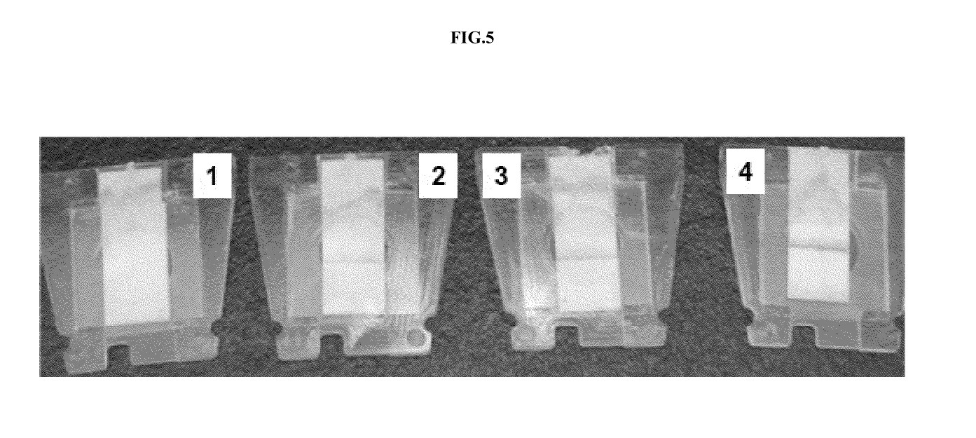

Experimental testing of anti-fPL (feline pancreatic lipase) conjugated colloidal gold showed good binding with fPL antigen panel. In the photos of FIG. 5, prototype Catalyst® Chemistry Analyzer slides were used to test the following four samples—from left to right: Panel 1, 0 ug/L fPL antigen; Panel 2, 1.7 ug/L fPL antigen, Panel 3; 3.4 ug/L PL antigen; and Panel 4, 6.2 ug/L fPL antigen. A clear direct correlation is observed; there is an increase in counts as a function of increasing concentration.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A device for conducting an assay to determine the presence or amount of an analyte in a fluid sample, comprising a flow path matrix that facilitates fluidic flow and a fluid impermeable barrier, wherein the flow path comprises:
(a) a first region on a top side of the barrier that facilitates fluid flow in a first direction and comprises a sample entry area,
(b) a second region on a bottom side of the barrier that facilitates fluid flow in a second direction substantially opposite the first direction and comprises a detection zone.

2. The device of claim 1, further comprising a third region in fluid communication with the first and second region.

3. The device of claim 1, wherein the flow path comprises a mobilizable conjugate reagent that binds to the analyte, wherein the conjugate reagent comprises a label or a first binding partner for a second binding partner comprising a label.

4. The device of claim 1, wherein the detection zone comprises an immobilized binding partner for the analyte or an immobilized analog of the analyte.

5. The device of claim 1, wherein the flow path has total length of less than 2 cm.

6. A device for conducting an assay to determine the presence or amount of an analyte in a fluid sample, comprising
(a) a flow path matrix that facilitates fluidic flow, wherein the matrix comprises a layer of matrix material partially circumscribing a planar fluid impermeable barrier having a top side and a bottom side, and wherein the matrix comprises a sample entry area on the top side of the fluid impermeable barrier and a detection zone on the bottom side of the fluid impermeable barrier.

7. The device of claim 6, wherein the flow path comprises a mobilizable conjugate reagent that binds to the analyte, wherein the conjugate reagent comprises a label or a first binding partner for a second binding partner comprising a label.

8. The device of claim 6, wherein the detection zone comprises an immobilized binding partner for the analyte or an immobilized analog of the analyte.

9. The device of claim 6, wherein the flow path has total length of less than 2 cm.

10. A method for conducting an assay to determine the presence or amount of an analyte in a fluid sample, comprising
(a) contacting the fluid sample with the device of claim 3,
(b) detecting the presence or amount of a signal associated with the label in the detection zone.

11. A method for conducting an assay to determine the presence or amount of an analyte in a fluid sample, comprising
(a) contacting the fluid sample with the device of claim 7,
(b) detecting the presence or amount of a signal associated with the label in the detection zone.

12. A method for conducting an assay to determine the presence or amount of an analyte in a fluid sample, comprising
(a) contacting the fluid sample with the device of claim 1 and conjugate reagent comprising a label or a first binding partner comprising a label,
(b) detecting the presence or amount of a signal associated with the label in the detection zone.

13. A method for conducting an assay to determine the presence or amount of an analyte in a fluid sample, comprising
(a) contacting the fluid sample with the device of claim 6 and conjugate reagent comprising a label or a first binding partner comprising a label,
(b) detecting the presence or amount of a signal associated with the label in the detection zone.

14. A test kit for conducting an assay to determine the presence or amount of an analyte in a fluid sample, the kit comprising a device according to claim 1, and a labeled specific binding reagent.

15. A test kit for conducting an assay to determine the presence or amount of an analyte in a fluid sample, the kit comprising a device according to claim 6, and a labeled specific binding reagent.

16. An apparatus for use in the detection of an analyte in a sample, the apparatus comprising a housing that retains the device of claim 1, a sample application window corresponding to the sample application area, and a detection window corresponding to the detection area.

17. The apparatus of claim 16, wherein the housing is configured to be inserted into an analyzer comprising a reader for reading a signal from detection zone through the detection window.
